(19) United States
(12) Patent Application Publication
(10) Pub. No.: US 2023/0005105 A1
(43) Pub. Date: Jan. 5, 2023

(54) RADIATION IMAGING SYSTEM, IMAGE PROCESSING METHOD, AND STORAGE MEDIUM

(71) Applicant: CANON KABUSHIKI KAISHA, Tokyo (JP)

(72) Inventor: Keisuke Arima, Kanagawa (JP)

(21) Appl. No.: 17/930,900

(22) Filed: Sep. 9, 2022

Related U.S. Application Data
(63) Continuation of application No. PCT/JP2021/008909, filed on Mar. 8, 2021.

(30) Foreign Application Priority Data
Mar. 16, 2020 (JP) 2020-045607

(51) Int. Cl. G06T 5/00; A61B 6/00

(52) U.S. Cl. CPC G06T 5/00; A61B 6/52; G06T 2207/10116; G06T 2207/30004

(57) ABSTRACT

A radiation imaging system comprises: an obtainment unit configured to obtain an image captured by radiation imaging; an image processing unit configured to generate a radiation image by applying image processing to the captured image; a display control unit configured to display, on a display unit, the radiation image with the image processing applied thereto; and a control unit configured to determine, based on an operation input, whether confirmation of the radiation image displayed on the display unit is complete.

### CROSS-REFERENCE TO RELATED APPLICATIONS

This application is a Continuation of International Patent Application No. PCT/JP2021/008909, filed Mar. 8, 2021, which claims the benefit of Japanese Patent Application No. 2020-045607, filed Mar. 16, 2020, both of which are hereby incorporated by reference herein in their entirety.

### BACKGROUND OF THE INVENTION

### Field of the Invention

The present invention relates to a radiation imaging system that irradiates a subject with radiation and captures a radiation image, an image processing method, and a storage medium.

### Description of the Related Art

Conventionally, there has been known a radiation imaging system including a radiation detection apparatus that irradiates a subject with radiation (for example, X-rays) and detects the intensity distribution of the radiation transmitted through the subject, thereby capturing a radiation image of a target.

For example, in radiation imaging such as front-of-chest imaging, reflection of a foreign object such as a necklace and disposable body warmer can be a problem. Particularly, re-imaging at a later date may be required due to reflection of a foreign object when it is not possible to confirm the presence of the reflection of the foreign object on a small monitor in radiation imaging in a medical examination car with limited space.

Attempts have been made to prevent oversight by applying image processing for enhancing a foreign object. Japanese Patent Laid-Open No. 2019-92857 describes a method of generating a plurality of images by applying a plurality of image processing operations set in advance to an image obtained by imaging.

However, the image processing of enhancing a foreign object is image processing that is effective in preventing oversight but not suitable for diagnosis. Therefore, operations are required to perform processing for reducing the influence of the image processing of enhancing a foreign object, and processing for preventing the image with the image processing of enhancing a foreign object applied thereto from being output to the outside. The burden of such operations becomes a problem in an examination workflow which requires efficient execution of radiation imaging.

The present invention has been made in consideration of the above-described problems, and provides a radiation imaging technique capable of efficiently executing radiation imaging while reducing the burden of operations for performing image processing required in radiation imaging.

### SUMMARY OF THE INVENTION

According to one aspect of the present invention, there is provided a radiation imaging system comprising: an obtainment unit configured to obtain an image captured by radiation imaging; an image processing unit configured to generate a radiation image by applying image processing to the captured image; a display control unit configured to display, on a display unit, the radiation image with the image processing applied thereto; and a control unit configured to determine, based on an operation input, whether confirmation of the radiation image displayed on the display unit is complete, wherein the image processing unit performs first processing of applying first image processing as the image processing, an if the control unit determines that confirmation of the radiation image is complete, performs second processing of applying, as the image processing, second image processing for generating a radiation image with a degree of processing reduced as compared to the first image processing.

Further features of the present invention will become apparent from the following description of exemplary embodiments with reference to the attached drawings.

### BRIEF DESCRIPTION OF THE DRAWINGS

The accompanying drawings, which are incorporated in and constitute a part of the specification, illustrate embodiments of the invention and, together with the description, serve to explain principles of the invention.

FIG. 1 is a view showing the overall configuration of a radiation imaging system according to an embodiment.

FIG. 2 is a view showing the arrangement of a control unit in the radiation imaging system according to the embodiment.

FIG. 3A is a view illustrating a display form of the radiation imaging system according to the embodiment.

FIG. 3B is a view illustrating a display form of the radiation imaging system according to the embodiment.

FIG. 3C is a view illustrating a display form of the radiation imaging system according to the embodiment.

FIG. 4 is a view illustrating a display form of the radiation imaging system according to the embodiment.

### DESCRIPTION OF THE EMBODIMENTS

Hereinafter, embodiments will be described in detail with reference to the attached drawings. Note, the following embodiments are not intended to limit the scope of the claimed invention. Multiple features are described in the embodiments, but limitation is not made to an invention that requires all such features, and multiple such features may be combined as appropriate. Furthermore, in the attached drawings, the same reference numerals are given to the same or similar configurations, and redundant description thereof is omitted. In the following embodiments and the appended claims, radiation includes, in addition to X-rays, α-rays, β-rays, γ-rays, and various kinds of particle beams.

### Configuration of Radiation Imaging System

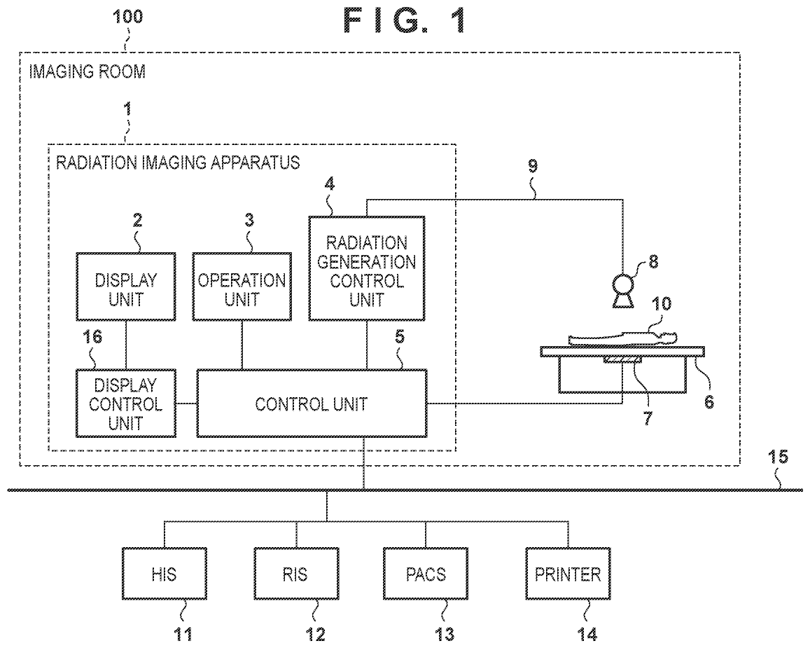

A radiation imaging system according an embodiment of the present invention will be described with reference to FIG. 1. FIG. 1 is a view showing a configuration example of a radiation imaging system according to an embodiment. As shown in FIG. 1, the radiation imaging system of this embodiment includes a radiation imaging apparatus 1, and an HIS (Hospital Information System) 11 that manages the progress of examination.

The radiation imaging system of this embodiment also includes an RIS (Radiology Information System) 12 that transmits an examination order to the radiation imaging apparatus 1. Further, the radiation imaging system of this embodiment is connected to a PACS (Picture Archiving and Communication Systems) 13 that manages a radiation image, and a printer 14 that prints out a radiation image.

The HIS 11 is a hospital management system, and includes a server that manages accounting information. When executing radiation imaging, an operator inputs an examination instruction using a terminal (input unit) of the HIS 11. Then, the HIS 11 transmits request information to a radiology department of the hospital which is the request destination of the radiation imaging. This request information is called an examination order. The examination order includes the department name of the request source, examination ID, examination items, patient information (subject information) of the subject (test object), and the like.

The RIS 12 is a radiology department information system. When the radiology department receives the examination order, the RIS 12 adds imaging information (imaging method, imaging conditions, image processing conditions, imaging portion information, imaging direction information, procedure information, and the like) of the radiation imaging to the examination order as imaging protocols, and transmits the examination order to the radiation imaging apparatus 1. The radiation imaging apparatus 1 executes radiation imaging in accordance with the received examination order. The radiation imaging apparatus 1 obtains a captured radiation image, generates examination information associating the radiation image and the examination order, and outputs the examination information together with the radiation image.

The PACS 13 is an image server mainly for image management. Using a high resolution monitor connected to the PACS 13, radiation image inspection work, detailed post-processing, and diagnostic work are performed. In this manner, the radiation image obtained by the radiation imaging apparatus 1 is transmitted to the PACS 13.

Examination execution information (image ID, imaging date and time, and the like) by the radiation imaging apparatus 1 is also transmitted to the HIS 11. The execution information transmitted to the HIS 11 is also used for, other than the progress management of the examination, an accounting process after the examination.

The radiation imaging apparatus 1, the HIS 11, the RIS 12, the PACS 13, and the printer 14 are connected via a network 15 formed from, for example, a LAN (Local Area Network) or a WAN (Wide Area Network).

Note that each of these apparatuses includes one or a plurality of computers. The computer is provided with, for example, a main control unit such as a CPU which is in charge of processing, and storage units such as a ROM (Read Only Memory) and a RAM (Random Access Memory). The computer may also be provided with a communication unit such as a network card, and input/output units such as a keyboard, a display, and a touch panel. These constituent elements are electrically connected by a bus and the like, and controlled by the main control unit executing programs stored in the storage unit.

As shown in FIG. 1, the radiation imaging apparatus 1 that executes radiation imaging is installed in an imaging room 100. A radiation generation control unit 4 that generates radiation, a radiation detector 7 that captures a radiation image by detecting the radiation transmitted through a subject 10, and an imaging table 6 are also installed in the imaging room 100.

The radiation imaging apparatus 1 includes a display unit 2 that displays a radiation image and various kinds of information, an operation unit 3 used by an operator to perform an operation, and a control unit 5 that controls respective components.

The radiation generation control unit 4 is connected to a radiation generator 8 via a cable 9, and controls the radiation generator 8 by setting radiation imaging conditions in the radiation generator 8. The radiation generator 8 functions as a radiation source that generates radiation. The radiation generator 8 is implemented by, for example, a radiation tube, and emits radiation toward the subject 10 (for example, a specific portion of the subject).

The radiation generator 8 can irradiate a desired irradiation range with radiation. A diaphragm (not shown) for shielding radiation is installed in the irradiation surface of the radiation generator 8. The operator can adjust the irradiation range of the radiation emitted from the radiation generator 8 by controlling the diaphragm that shields the radiation.

The radiation imaging system includes the radiation detector 7 that detects the radiation emitted from the radiation generator 8. The radiation detector 7 detects the radiation transmitted through the subject 10, and outputs image data corresponding to the radiation. Note that image data can also be called a radiation image.

More specifically, the radiation detector 7 detects the radiation transmitted through the subject 10 as electric charges corresponding to the transmitted radiation dose. For example, a direct conversion type sensor that directly converts radiation into electric charges, such as an a-Se sensor that converts radiation into electric charges, or an indirect type sensor using a scintillator such as a CsI scintillator and a photoelectric converting element such as an a-Si photoelectric converting element can be used as the radiation detector 7.

The radiation detector 7 A/D-converts the detected electric charges to generate image data, and accumulates it in a storage unit (not shown). The radiation detector 7 can add image information (image ID, imaging date and time, and image data transfer status) to the image data (radiation image), and transfer the image information to the radiation imaging apparatus 1 together with the image data.

The display unit 2 is implemented by, for example, a liquid crystal display or the like, and displays various kinds of information to an operator (for example, a radiographer, a doctor, or the like). The operation unit 3 is formed from, for example, a mouse, an operation button, and the like, and inputs various kinds of instructions from the operator to the respective components. Note that the display unit 2 and the operation unit 3 may be implemented as a touch panel integrating them.

The control unit 5 of the radiation imaging apparatus 1 is connected to the radiation detector 7 via a wireless LAN. Image data, control signals, and the like are transmitted and received between the control unit 5 and the radiation detector 7. That is, the image data stored in the radiation detector 7 by radiation imaging is output (transferred) to the control unit 5 via the wireless LAN.

### Description of Radiation Imaging System

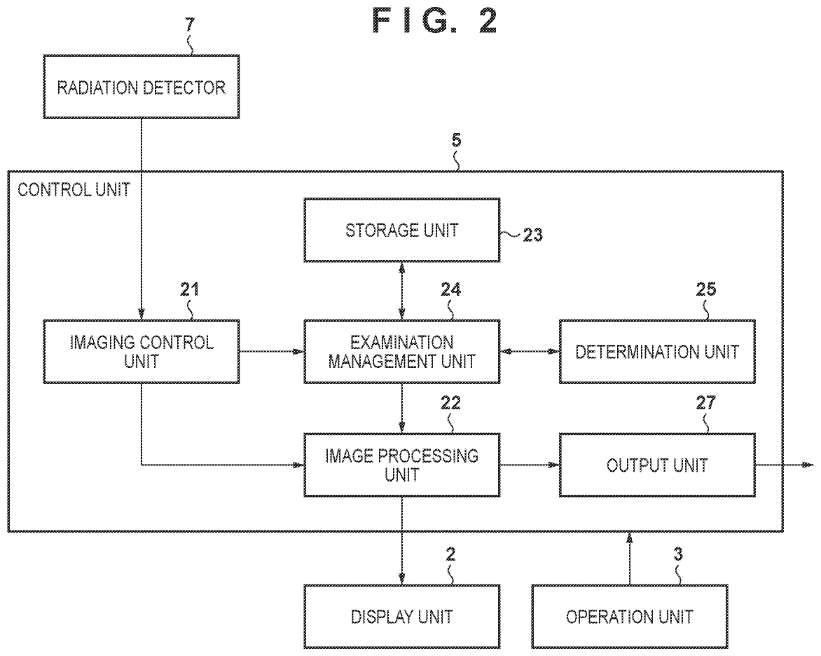

The functional configuration of a radiation imaging system according to an embodiment will be described in detail with reference to FIG. 2. The radiation imaging apparatus 1 includes the control unit 5 that generates an image by performing image processing on a radiation image output from the radiation detector 7. The control unit 5 has an application function that operates on a computer. The control unit 5 can control an operation of the radiation detector 7, and output a radiation image or a graphical user interface (GUI) to the display unit 2.

The control unit 5 includes an imaging control unit 21 that performs imaging control of the radiation detector 7, an image processing unit 22 that performs image processing on a radiation image obtained by imaging, and a storage unit 23 that stores a radiation image output from the radiation detector 7 and various kinds of information such as an examination order, imaging protocols, and imaging method.

The control unit 5 also includes an examination management unit 24 that manages examination information associating a radiation image and an examination order and imaging protocols, a determination unit 25 that determines the necessity of re-imaging from the radiation image and additional information, and an output unit 27 that outputs a generated image object to the outside.

The storage unit 23 stores the examination information (radiation image and examination order) and imaging protocols managed by the examination management unit 24, the imaging method, the radiation image output from the radiation detector 7, and various kinds of information necessary for examination management. The storage unit 23 also stores the imaging protocols associated with the examination order together with identification information for identifying the imaging protocols.

The examination management unit 24 manages the imaging protocols defining the imaging information (imaging method, imaging conditions, image processing conditions, imaging portion information, imaging direction information, and procedure information) of the radiation imaging and the like associated with the examination order. For example, when generating examination information in the radiation imaging apparatus 1, the examination management unit 24 can create new examination information by associating the subject information input from the operation unit 3 with the imaging protocols.

On the other hand, when the RIS 12 requests examination, the examination management unit 24 uses the identification information of the imaging protocols associated with the received examination order, thereby extracting the imaging protocols stored in the storage unit 23. The examination management unit 24 can create new examination information by associating the extracted imaging protocols with the examination order. The newly created examination information is stored in the storage unit 23.

The imaging control unit 21 transmits, to the radiation detector 7, a transfer request signal requesting transfer of a radiation image accumulated in the radiation detector 7, and receives the radiation image from the radiation detector 7. The imaging control unit 21 manages the received radiation image together with radiation detector information of the radiation detector 7. Further, the imaging control unit 21 associates the radiation image with the examination order and imaging protocols managed by the examination management unit 24.

The image processing unit 22 performs image processing on the radiation image using the imaging protocols, image data (radiation image), and image information obtained from the imaging control unit 21. The image processing unit 22 and a display control unit 16 display, on the display unit 2, the radiation image with image processing (first image processing and second image processing to be described later) applied thereto. Alternatively, the image processing unit 22 outputs the radiation image with image processing (second image processing) applied thereto to the outside from the output unit 27. The image processing unit 22 can perform image processing for adjusting an image itself, such as brightness/contrast, foreign object enhancement, and skin line enhancement. Further, the image processing unit 22 can also perform processing such as cutting out and annotating the adjusted radiation image.

The determination unit 25 determines the necessity of re-execution of radiation imaging based on the image data (radiation image) obtained from the imaging control unit 21 and the information (at least one of the imaging protocols and image information) set attached to the radiation image. The determination unit 25 determines reflection of a foreign object and the like from the image data (radiation image) and the information set attached to the radiation image.

The determination result of the determination unit 25 is transmitted to the examination management unit 24. The examination management unit 24 controls the image processing unit 22 based on the determination result of the determination unit 25.

If the determination result indicates “necessary”, that is, if it is determined that re-execution of radiation imaging is necessary based on the determination result, the examination management unit 24 controls the image processing unit 22 so as to apply enhancement processing (foreign object enhancement processing or skin line enhancement processing) as the first image processing, and prompts the operator to confirm the necessity/unnecessity of re-imaging.

If the determination result of the determination unit 25 indicates “unnecessary”, that is, if it is determined that re-execution of radiation imaging is unnecessary based on the determination result, the examination management unit 24 controls the image processing unit 22 so as to apply the second image processing with the reduced degree of processing of enhancement processing (foreign object enhancement processing or skin line enhancement processing), as in a case in which image confirmation is completed.

An example of the configuration of a radiation imaging system according to an embodiment of the present invention has been described above. Note that the configuration shown in FIG. 1 is merely an example, and can be appropriately changed. For example, although various kinds of apparatuses are connected to the radiation imaging apparatus 1 via the network 15, the radiation imaging apparatus 1 need not always be connected to these apparatuses. The diagnostic image may be output to a portable medium such as a DVD and input to various kinds of apparatuses via the portable medium. In addition, this network 15 may be formed as a wired network or may be partially formed by a wireless signal transmission path.

### Imaging Processing

Next, a processing procedure of capturing a radiation image along the sequence of an examination by the radiation imaging system shown in FIG. 1 will be described.

First, patient information and examination order are input to the radiation imaging apparatus 1 upon receiving an examination request document or an examination request from the RIS 12. The patient information includes a patient name, a patient ID, and the like. The examination information includes imaging information defining the contents of the imaging to be executed for the patient.

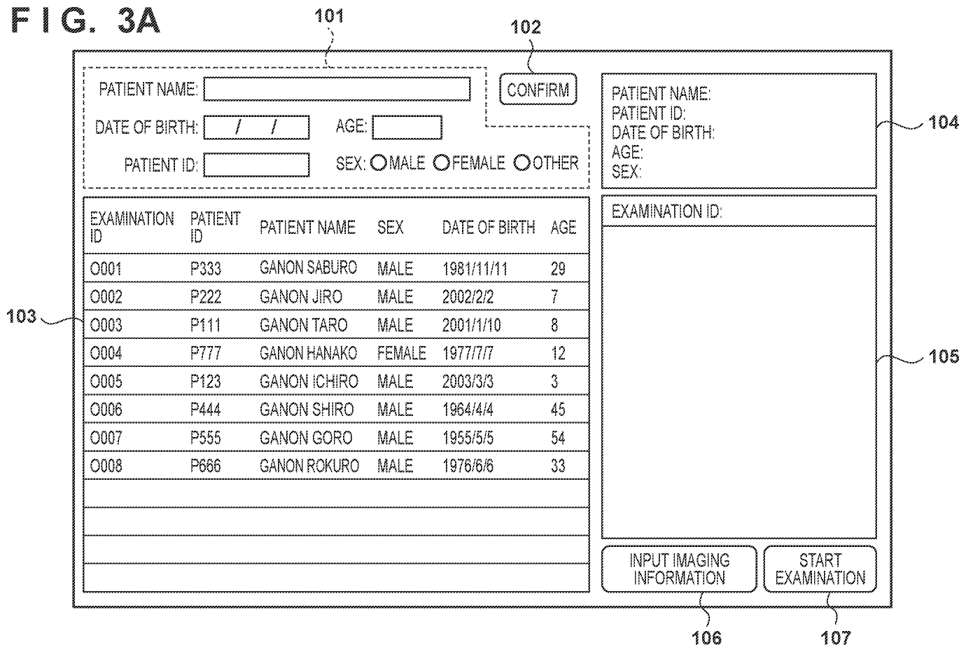

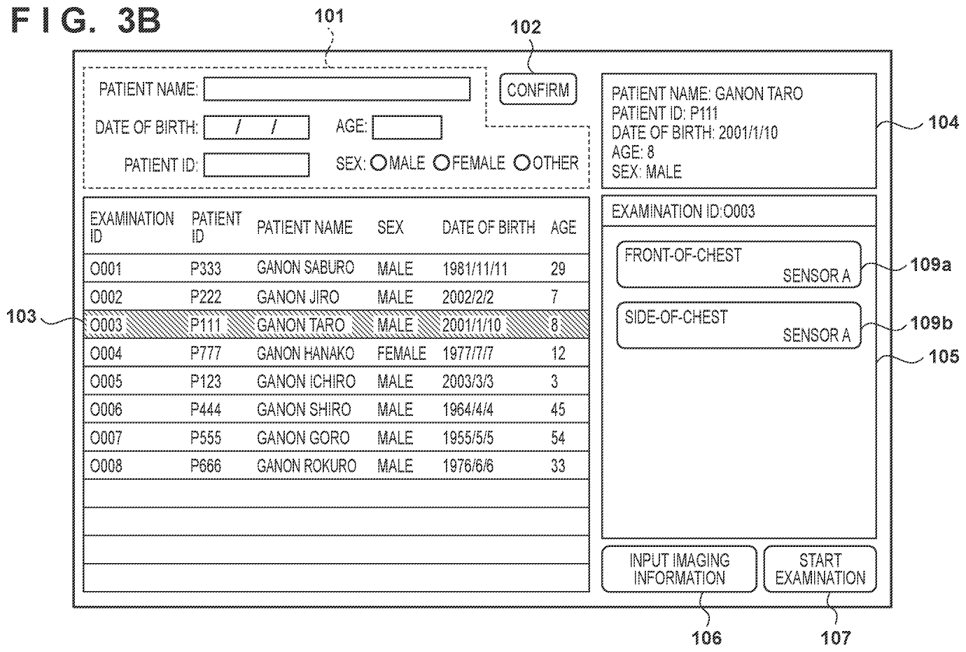

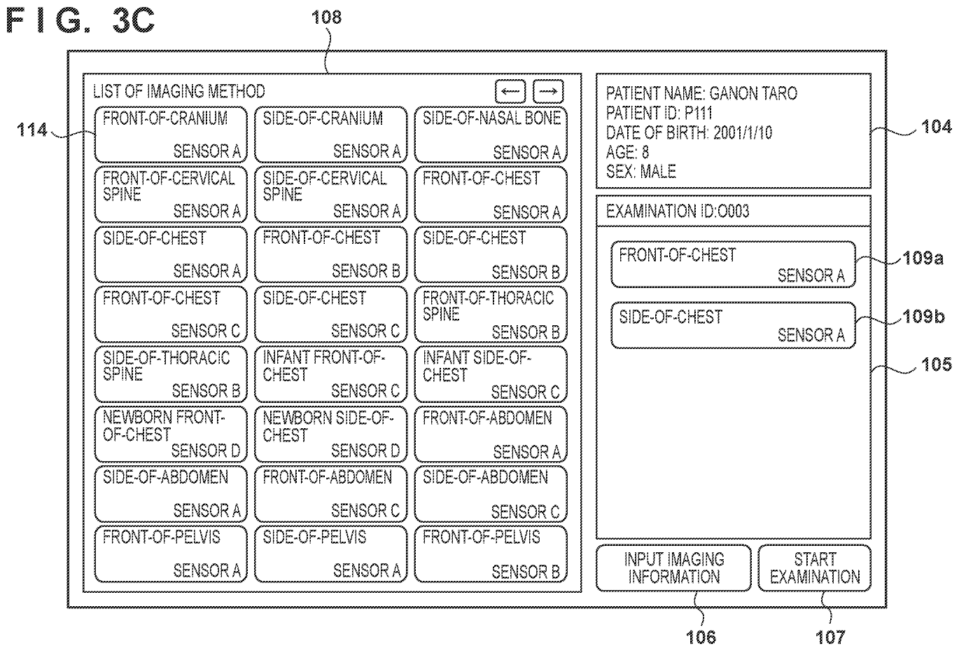

The radiation imaging apparatus 1 displays, by the control of the display control unit 16, a new examination input screen on the display unit 2 as shown in FIG. 3A. As shown in FIG. 3A, the new examination input screen includes a patient information input region 101, a patient information confirmation button 102, and a requested examination list 103. The new examination input screen shown in FIG. 3A also includes a patient information display region 104, an imaging information display region 105, an imaging information input button 106, and an examination start button 107.

Examination orders received from the RIS 12 are aligned and displayed as a list on the requested examination list 103. When one of the examinations is selected from the requested examination list 103, patient information (patient ID, patient name, date of birth, etc.) corresponding to the patient selected from the requested examination list 103 is displayed in the patient information display region 104 as shown in FIG. 3B. An examination ID is displayed in the imaging information display region 105, and imaging information corresponding to the examination ID is displayed in a region below the examination ID. As has been described above, the imaging information is information added to the examination order as imaging protocols. The display control unit 16 displays the imaging information received from the RIS 12 in the imaging information display region 105.

In the display example shown in FIG. 3B, imaging information buttons 109 (a front-of-chest button 109a and a side-of-chest button 109b) corresponding to the respective pieces of imaging information are displayed. When the imaging information input button 106 is input, the display control unit 16 displays an imaging information input region 108 as shown in FIG. 3C such that imaging information can be additionally selected from the imaging information input region 108. In the display example shown in FIG. 3C, a plurality of imaging method selection buttons 114 are displayed in the imaging information input region 108. An imaging method can be added by selecting one of the imaging method selection buttons 114. The display control unit 16 performs display control such that the added imaging method is aligned and displayed with the front-of-chest button 109a and the side-of-chest button 109b in the imaging information display region 105. Each imaging method is associated with the identification information (ID) capable of identifying the imaging method.

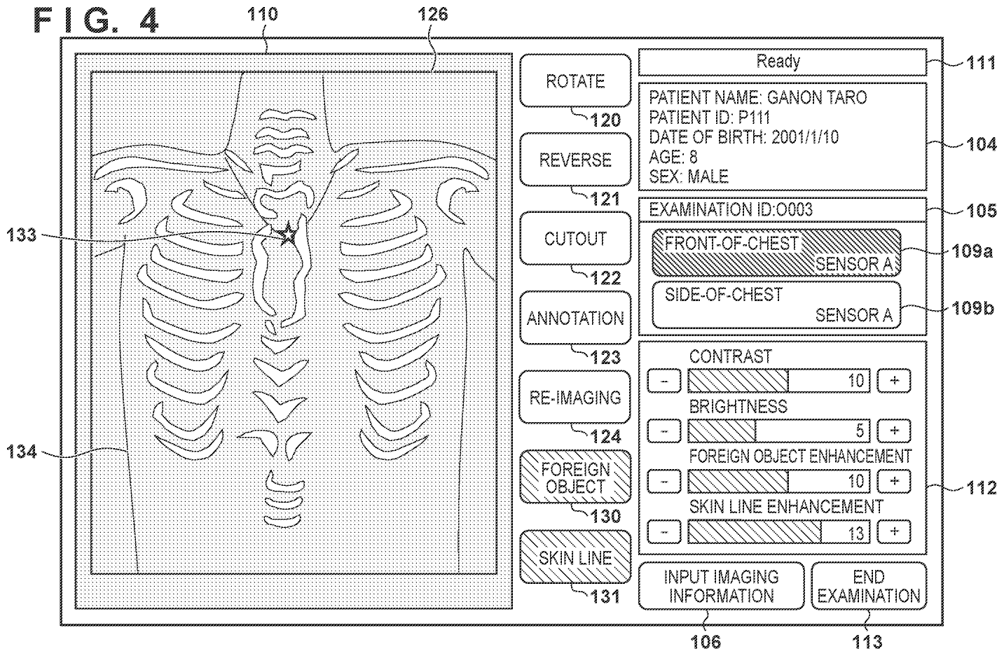

The operator presses the examination start button 107 after confirming the patient information and the imaging method. This confirms the examination to be performed. In accordance with the pressing of the examination start button 107, the display control unit 16 of the radiation imaging apparatus 1 displays an imaging screen as shown in FIG. 4 on the display unit 2. The imaging screen is a screen used during imaging.

The imaging screen as shown in FIG. 4 is configured to basically include the same display regions as those described in the new examination input screen in FIG. 3A. As shown in FIG. 4, the display control unit 16 displays, on the display unit 2, an image display region 110, a message region 111, an image processing setting region 112, and an examination termination button 113 as the display regions that are to be newly added.

When the imaging screen is displayed, the imaging information button (front-of-chest button 109a) which is arranged in the uppermost portion in the imaging information display region 105 is in a selected state by default. Along with this, the control unit 5 of the radiation imaging apparatus 1 transmits, to the radiation generation control unit 4, the imaging conditions (tube voltage, tube current, irradiation time, and the like) set in correspondence with the imaging method button (imaging method). The control unit 5 then controls the radiation detector 7 in accordance with the imaging conditions set in correspondence with the imaging method button (imaging method) to prepare for imaging. When the preparation for imaging is complete, the control unit 5 of the radiation imaging apparatus 1 changes the state of the radiation imaging apparatus 1 to an imaging enable state. At this time, the display control unit 16 performs display control so as to display a “Ready message” indicating the imaging enable state in the message region 111.

Next, the operator confirms the imaging method, performs imaging settings, and performs positioning of the patient. When a series of imaging preparation operations has been completed, the operator presses an irradiation instruction unit (radiation irradiation switch (not shown)) after confirming the imaging enable state by referring to the message region 111. In accordance with this, the radiation imaging apparatus 1 causes the radiation generator 8 to emit radiation toward the object (specific portion of the patient), and causes the radiation detector 7 to detect the radiation transmitted through the object. As a result, a radiation image is captured.

After imaging is complete, the control unit 5 of the radiation imaging apparatus 1 obtains the radiation image (to be also referred to as the captured image hereinafter) from the radiation detector 7, and the image processing unit 22 generates a radiation image by applying image processing to the obtained captured image based on predetermined image processing conditions. The predetermined image processing conditions are set in advance in correspondence with the imaging method and imaging conditions in the imaging protocols added to the examination order. Based on the set image processing conditions, the image processing unit 22 performs image processing on the radiation image obtained by imaging.

When the image processing ends, the image processing unit 22 and the display control unit 16 of the radiation imaging apparatus 1 display, in the image display region 110 of the display unit 2, the radiation image with the image processing applied thereto. The control unit 5 determines, based on an operation input, whether confirmation of the radiation image displayed on the display unit 2 is complete. The image processing unit 22 performs first processing of applying first image processing as the image processing and, if the control unit 5 determines that confirmation of the radiation image is complete, performs second processing of applying, as the image processing, second image processing for generating a radiation image with the degree of processing reduced as compared to the first image processing.

The image processing unit 22 applies the first image processing (for example, enhancement processing such as foreign object enhancement processing or skin line enhancement processing) to generate a radiation image with the degree of processing enhanced as compared to the second image processing. The image processing unit 22 applies the second image processing to the captured image as image processing common to the first processing and the second processing. The image processing unit 22 does not perform the first image processing (enhancement processing) in the second processing but applies the second imaging processing in the second processing, thereby generating a radiation image having no influence of the first image processing. For example, the image processing unit 22 can apply image processing regarding brightness and contrast as the second imaging processing, and apply image processing regarding enhancement processing such as foreign object enhancement and skin line enhancement as the first image processing.

If the operator wants to change the image processing of the captured image, he/she can change the image processing parameter by operating a button for contrast, foreign object enhancement, or the like provided in the image processing setting region 112. If a foreign object button 130 or a skin line button 131 is selected by an operation of the operation unit 3 by the operator, the image processing unit 22 selects the foreign object button 130 or the skin line button 131 to set it in an ON state (selected state). Both the foreign object button 130 and the skin line button 131 may be set in the selected states, or one of the foreign object button 130 and the skin line button 131 may be set in the selected state.

When the foreign object button 130 or the skin line button 131 is set in the ON state (selected state), the image processing unit 22 validates the enhancement processing corresponding to foreign object enhancement or skin line enhancement. If the image processing parameter in the image processing setting region 112 is changed while the foreign object button 130 or the skin line button 131 is set in the ON state (selected state), the image processing unit 22 performs image processing (first image processing) of changing the degree of enhancement in the captured image based on the change of the image processing parameter, and displays the captured image with the degree of enhancement changed in the image display region 110.

On the other hand, if the foreign object button 130 and the skin line button 131 are unselected and set in the OFF states (unselected states), the image processing unit 22 invalidates corresponding enhancement processing as the second image processing, and displays the captured image with corresponding enhancement processing invalidated in the image display region 110. Both the foreign object button 130 and the skin line button 131 may be set in the unselected states, or one of the foreign object button 130 and the skin line button 131 may be set in the unselected state.

Note that as setting of enhancement processing, it is possible to set such that enhancement processing is automatically applied to the captured image as the first image processing by setting the foreign object button 130 and the skin line button 131 in the ON states (selected states) immediately after imaging.

When the foreign object button 130 is selected to apply foreign object enhancement processing as the first image processing, for example, an image in which a foreign object 133 such as a necklace or an accessary is enhanced is displayed as shown in FIG. 4. The image processing unit 22 generates a radiation image by applying foreign object enhancement processing to the captured image obtained by radiation imaging, and displays the radiation image. With this, the operator can confirm the presence/absence of reflection of a foreign object. If it is confirmed from the radiation image that there is no reflection of a foreign object, the operator confirms that re-imaging is unnecessary.

When the skin line button 131 is selected to apply skin line enhancement processing as the first image processing, for example, an image is displayed in which a skin line 134, which is a boundary between a region where the radiation transmitted through the subject 10 is detected and a region where the radiation is detected without being transmitted through the subject 10, is enhanced. The image processing unit 22 generates a radiation image by applying skin line enhancement processing to the captured image obtained by radiation imaging, and displays the radiation image. With this, the operator can check whether the radiation irradiation range was appropriate with reference to the position of the skin line. If it is confirmed from the radiation image that the position of the skin line is appropriate, the operator confirms that re-imaging is unnecessary.

If the operator wants to change the cutout region of an output image, he/she can operate a cutout button 122, a cutout frame 126, and the like via the operation unit 3 to designate the desired cutout region. When a character string which is to serve as diagnostic information is to be added to the radiation image, the operator operates an annotation button 123 or the like via the operation unit 3 to superimpose the character string on the radiation image. If the orientation of the image is not suitable for diagnosis, the operator can operate a rotate button 120, a reverse button 121, or the like via the operation unit 3 to perform geometric conversion for rotating or reversing the radiation image. As described above, the operator can perform additional image processing on the radiation image displayed in the image display region 110.

When the imaging information button (side-of-chest button 109b: designation unit), which designates execution of radiation imaging to obtain a captured image according to the imaging protocol set next to the radiation image (captured image) captured by the radiation detector 7, is pressed to input a radiation imaging execution instruction, the control unit 5 controls the radiation detector 7 in accordance with the imaging conditions corresponding to “side-of-chest, sensor A” set for the imaging information button (side-of-chest button 109b) to prepare for imaging.

If execution of the radiation imaging is designated by the imaging information button (side-of-chest button 109b: designation unit) as the operation input, the control unit 5 determines that image confirmation of the radiation image with the image processing applied thereto is complete. That is, the control unit 5 determines that image confirmation of the radiation image (the image corresponding to the imaging information button 109 (front-of-chest button 109a)) obtained by the preceding imaging is complete.

It is also possible that, if radiation irradiation is instructed as the operation input by the irradiation instruction unit (radiation irradiation switch) which instructs to perform radiation irradiation to obtain a captured image according to the imaging protocols set next to the radiation image (captured image) captured by the radiation detector 7, the control unit 5 determines that image confirmation of the radiation image with the image processing applied thereto is complete.

If image confirmation is complete, the image processing unit 22 changes the degree of enhancement so as to reduce the degree of processing (influence of processing) in the enhancement processing (foreign object enhancement processing or skin line enhancement processing). For example, the image processing unit 22 changes setting of the parameter for foreign object enhancement from 10 to 2 in the image processing setting region 112, and changes setting of the parameter for skin line enhancement from 13 to 1. By changing the numerical value of the parameter to a smaller value, the degree of processing (influence of processing) in the corresponding enhancement processing can be reduced.

The parameter indicating the degree of processing in the enhancement processing after image confirmation can be set for the imaging protocol in advance. It is also possible to make setting so as to invalidate the enhancement processing (foreign object enhancement processing or skin line enhancement processing) after image confirmation. To invalidate the enhancement processing is to set the numerical value of the parameter to 0. Along with the invalidation of the enhancement processing (foreign object enhancement processing or skin line enhancement processing), the foreign object button 130 or the skin line button 131 is set in the OFF states.

The operator repeats the procedure described above, and the control unit 5 executes radiation imaging specified by all the examination IDs displayed in the imaging information display region 105. If radiation imaging is complete for all the examination IDs, the operator presses the examination termination button 113 (termination instruction unit) which terminates examination by radiation imaging. If termination of examination is instructed by the examination termination button 113 (termination instruction unit) as the operation input, the control unit 5 determines that confirmation of the last radiation image with the image processing applied thereto is complete. If image confirmation is complete, a series of examinations ends accordingly. At this time, the image processing unit 22 determines that image confirmation of the last imaging is complete, and changes the degree of enhancement so as to reduce the degree of processing (influence of processing) in the enhancement processing (foreign object enhancement processing or skin line enhancement processing).

The output unit 27 of the radiation imaging apparatus 1 outputs the radiation image (image object) with the second image processing applied thereto and the examination information, imaging conditions, and the like added thereto as the additional information by the control unit 5 to, for example, the PACS 13, the printer 14, or a ROM in the self-apparatus. For the foreign object enhancement processing and the skin line enhancement processing, the radiation image (image object) with the processing (second image processing) having the reduced degree of processing applied thereto in the procedure described above is output to the outside. The radiation imaging apparatus 1 displays the new examination input screen again.

According to the embodiment described above, it is possible to efficiently execute radiation imaging while reducing the burden of operations for performing image processing.

### Other Embodiments

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

1. A radiation imaging system comprising:
an obtainment unit configured to obtain an image captured by radiation imaging;
an image processing unit configured to generate a radiation image by applying image processing to the captured image;
a display control unit configured to display, on a display unit, the radiation image with the image processing applied thereto; and
a control unit configured to determine, based on an operation input, whether confirmation of the radiation image displayed on the display unit is complete,
wherein the image processing unit performs first processing of applying first image processing as the image processing, and
if the control unit determines that confirmation of the radiation image is complete, performs second processing of applying, as the image processing, second image processing for generating a radiation image with a degree of processing reduced as compared to the first image processing.

2. The radiation imaging system according to claim 1, wherein
the image processing unit applies the second image processing to the captured image as image processing common to the first processing and the second processing.

3. The radiation imaging system according to claim 1, wherein
the image processing unit applies the second imaging processing in the second processing to generate a radiation image having no influence of the first image processing.

4. The radiation imaging system according to claim 3, wherein
the image processing unit does not perform the first image processing in the second processing.

5. The radiation imaging system according to claim 1, wherein
the image processing unit applies the first image processing to generate a radiation image with a degree of processing enhanced as compared to the second image processing.

6. The radiation imaging system according to claim 1, further comprising
a designation unit configured to designate execution of the radiation imaging to obtain a captured image according to an imaging protocol set next to the captured image,
wherein if execution of the radiation imaging is designated by the designation unit as the operation input, the control unit determines that confirmation of the radiation image with the image processing applied thereto is complete.

7. The radiation imaging system according to claim 1, further comprising
an irradiation instruction unit configured to instruct to perform radiation irradiation to obtain a captured image according to an imaging protocol set next to the captured image,
wherein if the radiation irradiation is instructed by the irradiation instruction unit as the operation input, the control unit determines that confirmation of the radiation image with the image processing applied thereto is complete.

8. The radiation imaging system according to claim 1, further comprising
a termination instruction unit configured to terminate examination by the radiation imaging,
wherein if termination of examination is instructed by the termination instruction unit as the operation input, the control unit determines that confirmation of the radiation image with the image processing applied thereto is complete.

9. The radiation imaging system according to claim 1, further comprising:
a determination unit configured to determine a necessity of re-execution of radiation imaging by the obtainment unit based on the radiation image and information set attached to the radiation image; and
a management unit configured to control the image processing unit based on a determination result of the determination unit,
wherein the management unit
controls the image processing unit so as to apply the first image processing if it is determined that the re-execution of radiation imaging is necessary based on the determination result, and
controls the image processing unit so as to apply the second image processing if it is determined that the re-execution of radiation imaging is unnecessary based on the determination result.

10. The radiation imaging system according to claim 1, further comprising
an output unit configured to output the radiation image to an outside,
wherein the output unit outputs, to the outside, the radiation image with the second image processing applied thereto in the second processing.

11. An image processing method in a radiation imaging system that comprises
an obtainment unit configured to obtain an image captured by radiation imaging,
an image processing unit configured to generate a radiation image by applying image processing to the captured image,
a display control unit configured to display, on a display unit, the radiation image with the image processing applied thereto, and
a control unit configured to determine, based on an operation input, whether confirmation of the radiation image displayed on the display unit is complete, the method comprising:
performing image processing in which the image processing unit performs first processing of applying first image processing as the image processing and, if the control unit determines that confirmation of the radiation image is complete, performs second processing of applying, as the image processing, second image processing for generating a radiation image with a degree of processing reduced as compared to the first image processing.

12. The image processing method according to claim 11, wherein
in the performing the image processing, the second image processing is applied to the captured image as image processing common to the first processing and the second processing.

13. The image processing method according to claim 11, wherein
in the performing the image processing, the second imaging processing is applied in the second processing to generate a radiation image having no influence of the first image processing.

14. The image processing method according to claim 13, wherein
in the performing the image processing, the first image processing is not performed in the second processing.

15. The image processing method according to claim 11, wherein
in the performing the image processing, the first image processing is applied to generate a radiation image with a degree of processing enhanced as compared to the second image processing.

16. A non-transitory computer readable storage medium storing a program for causing a computer to execute a step in the method according to claim 11.
